(19) United States
(12) Patent Application Publication
(10) Pub. No.: US 2020/0306499 A1
(43) Pub. Date: Oct. 1, 2020

(54) METHODS, BEDWEAR AND BEDDING FOR AIDING SLEEP

(71) Applicant: Martin BREACH, Haydock (GB)

(72) Inventor: Martin BREACH, Haydock (GB)

(21) Appl. No.: 16/835,838

(22) Filed: Mar. 31, 2020

(30) Foreign Application Priority Data
Mar. 31, 2019 (GB) 1904519.4

(51) Int. Cl. A61M 21/02

(52) U.S. Cl. CPC A61M 21/02; A61M 2021/0022

(57) ABSTRACT

A method of aiding sleep includes a person seeking to sleep using a finger to touch and move the finger over at least part of a tactile region located on bedwear that the person is wearing, or on bedding present on a bed, in a manner that can promote sleep. Bed wear and bedding including such a tactile region is also provided.

### CROSS REFERENCE TO RELATED APPLICATIONS

This application claims priority from Great Britain Patent Application No. 1904519.4 filed on Mar. 31, 2019, the entire disclosure of which is incorporated herein by reference.

### BACKGROUND

Many people have difficulty in getting to sleep.

Much research in aiding sleep has been focused on improving the comfort of mattresses and pillows to help aid sleep.

For example, there has been a recent trend for mattresses and pillows to incorporate memory foam. This can mould to a person's contours as the person moves and can thus improve comfort.

However the provision of comfortable bedwear and bedding does not always provide significant improvements in sleep.

There can, for example, still be major problems with restlessness even if a person is comfortable in bed.

Although well-known relaxation techniques such as listening to soothing music, hypnotherapy, aromatherapy etc, can help, they are not always effective, and may be inconvenient to use and/or risk disturbing a sleeping partner.

There is therefore a need for alternative ways of promoting sleep.

### SUMMARY OF THE INVENTION

The present invention provides bedwear and bedding that can help a person in getting to sleep more quickly/more regularly/more easily than would otherwise be the case.

According to the present invention there is also provided a method of adding sleep whereby a person in bed uses a finger to touch and progressively focus over all or at least part of a tactile region or pattern located on bedwear that the person is wearing or that is present on bedding.

To promote a progressive focus, the tactile region or pattern desirably comprises one or more of the following: a spiral shape or pattern, a group of nested or concentric shapes or patterns, a series of shapes or patterns that get progressively smaller or larger in a given direction, a line arranged to provide a repeated motif or pattern, a shape that includes a plurality of branches, a generally fractal shape, a series of shapes or patterns having a common theme with variations, or a repeated pattern.

A person seeking to sleep can, without having to move their hand, move a finger a plurality of times over one or more shapes or patterns of the tactile region enabling and promoting a deeper and deeper focus on the component or components being explored, until the person reaches a state where they are more likely to drift to sleep. Desirably the finger maintains contact with a given shape or pattern, whilst it moves over it or at least over a part thereof.

For example an individual seeking to sleep can move the finger along a spiral shape or pattern once or several times, to explore with their sense of touch the small elements comprising that shape or pattern.

Movement can be along a given direction or path. Thus the person can use the sense of touch to move the finger in a given manner whilst in contact with at least part of the tactile region.

It is possible that two or more fingers are used to trace a given path. Thus the term “a finger” does include the plural, although it is preferred that a single finger be used.

The term “finger” also includes a thumb.

It is desired that if there is a single element is provided, this may for example be no wider than the range of movement achievable by the tip of a single finger of a user whilst the hand remains still. Single elements may be combined in such a way that they in such a way that different components can be explored by different fingers of the same hand.

It is desired that if a path or pathway is provided it is shaped to be traced by a single finger rather than several fingers. It may for example be no wider than a finger of a user. Average dimensions are available in most countries for people of a given age/age range.

A path or pathway to be traced by a finger may therefore be one that is no wider than the mean finger width of an adult population of a country. Thus it may be less than 2 cm wide, or less than 1.5 cm wide, at the widest point.

This is however simply preferred rather than essential.

Whatever the width of any path/pathway (if one is provided) it can be formed by a variety of shapes.

For example the person may move finger along a spiral path/pathway from the outermost to the innermost coil of the spiral and/or vice versa.

If desired the person may repeatedly move the finger along a path whilst maintaining contact with it. This can be done in a given direction and/or in a reverse direction.

Preferably the method includes at least two, at least three, at least five, or at least ten repetitions and/or reversals of a given movement.

It is important to note that, although many methods for aiding to sleep have been proposed in the prior art, relatively few utilise the sense of touch.

Those that do often rely upon pressure being applied to the person by another person or a device.

Thus, for example, another person may give a person seeking to sleep a massage. This can relax the person and may sometimes aid sleep. There are also various devices that can be used to mimic massage techniques. For example, a person seeking to sleep can sit still in a specialised chair or lie down on a bed that can provide an automated massage

In the above methods the person seeking to sleep is relatively inactive

This contrasts with the present invention whereby the person is active in moving a finger over a tactile region and needs to concentrate and focus whilst doing so. The present invention can be considered counterintuitive in that it might be considered that a person concentrating on moving his/her finger over a tactile region of bedwear or bedding would be deterred from getting to sleep. However, a progressive concentration on finer and finer details of what the exploring finger can distinguish is a process that leads to a deeper and deeper mental focus, that can naturally lead on to sleep. This method can be very effective.

The human sense of touch is capable of detecting very fine levels of detail.

Static touch can feel something as small as one fifth of a millimetre (half the width of an eyelash). That is 200,000 nanometres.

In haptics engineering, the casual usable dynamic tactioception discrimination limit for humans is considered to be around 9 microns. Thus, for example, if there is a really smooth surface with a 10×10×10 micron cubical bump, it can often be detected when a person slides (untreated/unprepared) fingers over it even if the person is only mildly attentive. The usable static feature discrimination limit, feature when skin is statically pressed against the feature (different mechanoreceptors are involved in detecting such features) is around 100 microns (around the width of an adult human hair).

Without being bound by theory it is thought that movement of the finger whilst it is in contact with the tactile region may be useful in distracting the person from thoughts or worries that may prevent sleep.

Repeating and/or reversing the movement can also gradually increase the depth of mental focus and the onset of sleep.

A useful analogy here is that it is sometimes recommended that a person get out of bed and perform a routine task, such as completing a jigsaw. This puts the person in a different environment from where they may have difficulty in getting to sleep and can help distract the mind. However the person would still normally need to get out of bed. Furthermore a light would normally be needed to be switched on to help the person see and connect pieces of the jigsaw.

The light can affect melatonin levels and thereby reduce the likelihood of the person getting to sleep relatively quickly, as can the activity in getting out of and subsequently back into bed.

An advantage of the present invention is that it can be performed in a dark environment, without the need for any illumination. Thus there is no need for the tactile region to be visible.

A further advantage of the method of the invention is that it can be performed by a person seeking to sleep by themselves without external assistance from others.

Thus there is no need for any therapy or treatment to be practised upon the person by anyone else. The invention can therefore be easily used by people who do not wish to seek treatment from others for poor sleep patterns. It is thus different from sleep therapy techniques applied by others to a patient such as hypnotherapy, massage etc.

It is envisaged that by using the present invention a person seeking to sleep may fall asleep within 60 minutes, within 45 minutes or within 30 minutes of commencing the method of the present invention. More preferably, the person may fall asleep within 30 minutes or 20 minutes, or 10 minutes or even 5 minutes of commencing the method.

It is of course still possible that the method will not work on every occasion or for every person.

It may also take some practice for a given person who can benefit from the method to get accustomed to it.

In many cases however major improvements in one or more of the following

a) the time taken to get to sleep
b) the quality of sleep
c) the regularity of sleep patterns.

The method of the invention has significant industrial application in that it can be used anyone to improve sleep and thereby to improve efficiency at work.

This applies whether or not a person seeking to sleep has a sleep disorder.

Thus, for example, it can be used by workers to help improve their alertness, readiness to work the following day, etc.

The work may be any type of work. It includes industrial work and/or any work where a significant level of alertness is needed.

Thus the invention includes a method of providing a more efficient worker comprising the step of using the present invention to improve sleep prior to work.

There is further industrial application in that courses in training a person to sleep and thereby to be more efficient at work/when not asleep can be provided.

The invention can also be used to improve performance in sport or training, given that improved sleep has been shown to boost performance.

The invention can be used to improve the quality and/or amount of sleep. In particular, it can be used to improve the amount of R.E.M. sleep.

It can also be beneficial simply by allowing a person to get to sleep more quickly than would otherwise be the case so that the person can organise his/her time more efficiently. Thus, even if the person gets the same amount of sleep as usual, the present invention allows less time to be spent in bed because the person can get to sleep more quickly.

The invention is particularly useful for shift workers who have irregular work and sleep patterns and may find it difficult to get to sleep.

However it can be used for anyone seeking to get to sleep more quickly/more easily.

It is equally suitable for children to use as for adults, and is wholly safe without any risk of adverse effects.

As indicated earlier, the tactile region can be located within the expected reach of an average individual's hand on bedding. For example it can be located on a pillow slip, a duvet cover, a bedsheet, a blanket, a quilt or even on a mattress.

It is preferred however that it is located on the bedwear that the person seeking to get to sleep is wearing.

This avoids any need to change the design of bedding.

Another important point is that the person seeking to sleep will have the bedwear on and thus readily available and will not need to fumble around to find a tactile region located on bedding (which may have moved anyway as a person moves in bed).

If the tactile region is located on bedwear it is preferred that it is located at a given region of the bedwear.

More particularly it is preferred that it only located as a given region.

The region may, for example be a sleeve, cuff, collar, leg, side, side, pocket and/or torso area of the bedwear.

It is preferred that the tactile region is located at a front or side outer face of the bedwear.

The bedwear may be one piece or multiple piece. For example it may be in the form of pyjamas, shorts, a tee shirt, a dressing gown, a night gown, a night dress, a chemise, leggings, a night shirt, long johns, a “onesie”, etc. The term “bedwear” includes both nightwear and loungewear.

It is preferred that it is not in the form of daywear, given that bedwear is generally designed for aiding sleep and for comfort while in bed.

The tactile region is preferably easily accessible to a person wearing the nightwear whilst in the dark in bed and aiming to get to sleep.

The tactile region should provide a shape or pattern that is raised or recessed so that it can be easily located in the dark by touch.

It is generally not sufficient therefore that a shape/pattern is merely printed.

There are many shapes/patterns found on bedwear or bedding that have been printed onto fabric so that they can be seen in daylight, but that a person seeking to sleep would simply be unable to detect merely by touch. Thus it is preferred that shapes/patterns of the present invention that can be used in helping a person get to sleep are not printed ones, unless the printing provides a raised/recessed shape or pattern that can be detected by touch in a method of the present invention, or unless the shapes/patterns are anyway raised/recessed anyway and printing does not change this. (For example, a patch in the form of a spiral shape may be sewn onto bedding or bedwear to provide a raised spiral pathway that a finger can feel move along to help aid sleep. The patch may include a printed colour/pattern on it, but this is optional.)

Desirably the shape/pattern is raised or recessed by at least 0.1 mm, 0.2 mm, 0.3 mm, 0.4 mm 0.5 mm, at least 1 mm, or at least 2 mm relative to adjacent material.

More desirably, it will be raised or recessed by at least 3 mm, at least 4 mm at least 5 mm relative to adjacent material.

This can be done by known techniques in the art of fabric and clothing manufacture

For example sewing, embroidery, patching. knitting and/or crocheting can be used.

Any other techniques can be used as long as the shape(s)/pattern(s) can readily be detected by touch.

The tactile region may, for example, be stuck in place by adhesive, may be formed by moulding or stamping techniques, or may be formed by embossing techniques may be used, etc.

It is even possible to releasably secure a given tactile region in place (e.g. by using hook and eye fasteners such as Velcro™, or by using other releasable securing means, including other releasable fasteners, releasable press studs, clips, etc.)

More preferably, however, the tactile region is fixed in place rather than being releasably secured in place.

As explained above, the tactile region can include one or more shapes and/or patterns that can be detected by touch.

The terms “shape” and “pattern” includes both solid shapes/patterns and outlines thereof.

Outlines can help facilitate for nesting.

Thus a series of rings (outlines of circles/discs) can be nested within one another.

The outline need not be linear or indeed in the form of a line. It may provide a pathway for a finger to track. Thus for example a ring can be considered to provide an annular track/pathway that a finger can move along.

If a series of nested shapes is provided then a user may move a finger inwards and/or outwards to contact different nested elements in a manner.

Thus for example in the case of nested rings a finger might be used to trace around an outer nested ring and can then move inwardly to contact and then move around an inner ring.

A user is of course free to choose how to move a finger in contact with the tactile region in a manner that helps the user to get to sleep.

A shape can be raised or recessed by any desired means. Thus it can be provide via stitching, embroidery, a patch or by any other means.

It is preferred that the texture of the raised or recessed area is different from that of adjacent material. For example it may be of a significantly rougher or smoother texture so that it can be easily be distinguished by touch from the surrounding area.

As discussed above, the tactile region may provide a path for a finger to touch and follow.

Thus for example the region many be provided to allow a finger to trace a path in which it contacts progressively smaller components and/or a progressively finer/more complex level of detail as it moves along the path in a given direction.

In one embodiment the finger may start moving over an area made up of multiple threads but the number of threads then may reduce, e.g. to a single thread.

If desired a tapered tactile region may be provided. This is however not essential. The region may provide a path that is not tapered (e.g. of constant or irregular width or a path that branches out).

The tactile region may even comprise a fractal shape.

A fractal shape is sometimes considered to be a shape that exhibits “self similarity” as it is viewed at increasingly small scales. This means it has the same/similar patterns as it is magnified to an increasing level.

Another example is a fractal tree, whereby a trunk (or other moiety) splits into branches, each branch splits into further branches and so on.

A further example of a fractal shape is a fern-like structure, whereby each leaf of a fern looks like the fern itself and the same applies to parts of the leaf, to parts of those parts, etc.

The Barnsley fern is an example of a fractal shape.

Other fractal shapes are also useful in the present invention. This is because a finger can find further detail on increasingly small scales as it traces along the fractal shape.

A useful example here is the Koch snowflake. This can be considered as being formed from an equilateral triangle that has the central third of every line segment replaced with a pair of line segments that also form part of an equilateral triangle.

This procedure is iterated again and again to provide further levels of detail.

The outline of the Koch snowflake is sometimes known as the Koch curve (or von Koch curve). The Koch snowflake/curve is an example of a fractal with “exact” self-similarity, whereby exactly the same pattern/shape is repeated at various scales.

Other such fractal shapes include the Sierpinski triangle, the Sierpinski carpet, etc.

Exact self-similarity is preferred, although not essential, for fractal shapes used in the present invention.

The Mandelbrot set is an example of a fractal shape with so-called “quasi” self-similarity, whereby a shape/pattern is recognisably similar to, but not identical to, a corresponding shape/pattern at a different scale. Thus Mandelbrot satellites when viewed zooming in from a larger image are considered similar to, but not the same as, the larger image. Other fractal sets that have been used to form fractal shapes include the Julia set, and the Fatou set.

There are many other shapes useful in the present invention

For example there are space-filling curves that exhibit finer and finer detail at repeated iterations. These include the Hilbert curve, the Peano curve, the Dragon curve, etc.

Again these can be considered as fractals useful in the present invention

There are also random fractals that are based on stochastic rules.

Coastlines are sometimes given as examples of such random fractals.

Viewed at low magnification a coastline may be considered to have a given approximate length, but when viewed at increasingly finer detail the length can be considered to get larger and larger as more and more detail is shown. It may even approach infinity.

Of course, in practice, replicas of coastlines (or other replicas) provided on bedwear or bedding cannot produce fine detail to an infinite limit. They can however provide enough detail for a person seeking to sleep to be able to move a finger along them and find sufficient interest and variation to become diverted from thoughts that might prevent the person falling asleep. There should also be sufficient detail for the coastline to be recognisable by touch and/or by sight. (In practice, a person will normally have viewed bedding or bedwear before actually using it in a method of the present invention. Thus the sense of touch will normally be used to confirm a shape that the person is already aware of and to trace along it in a manner that may aid sleep.)

Preferred coastline replicas are ones that are generally recognisable.

A person based in Great Britain may therefore have a particular interest in tracing a finger along a replica of the coastline of Great Britain, or a part of said coastline.

It is possible for the tactile region to provide a replica of the outline of a country or other region, without including any part representing a coastline. This is because some countries have no coastlines.

Replicas of maps may also be provided (e.g. of a world map or of a map of a region). Here raised/recessed shapes (including outlines thereof) can represent countries or regions of interest such as mountainous areas, lakes and/or towns and cities.

Although fractals such as coastlines or country borders are sometimes be considered to have repeated levels of detail to an infinitely small scale, in practice it is not possible to reproduce an infinite level of detail in a manufactured article.

Indeed even in nature apparently fractal shapes will usually lose self-similarity at small scales. There is also the point that a person's sense of touch will not in any event be able to detect shapes/patterns at very small scales.

The term “simulated fractal” is often used to take into account the point that, although self-similarity may occur for several repetitions/levels of iteration, it can break down at a given level.

For the purposes of the invention, a simulated fractal is considered to be within the definition of “fractal”.

Furthermore, for the purposes of the present invention, a simulated fractal is considered to allow for self-similarity at a level of at least three repetitions/iterations. More preferably, there will be self-similarity at a level of at least four or five repetitions/iterations.

Thus, for example, in the case of the fern-like fractal a large the fern will be similar at a first level of magnification, at a second level of magnification and a third level of magnification. Similarly, if a fractal shape/pattern is formed by repeated branching, a simulated fractal would be considered to be formed after at least three levels of branching. A person seeking to sleep would have numerous routes/pathways that a finger could trace along a simulated fractal shape/pattern. This can be useful in getting a person to sleep because it can help distract the mind from other thoughts. The numerous pathways can also cause a person seeking to fall asleep after tracing a finger along a number of them.

Fractal based shapes or patterns are of course not limiting on the present invention but are simply examples of shapes/patterns that can be raised or recessed and used to provide the tactile region.

Thus a person seeking to sleep can use a finger to contact and move over one or more raised or shapes or patterns.

The shapes or patterns can be regular or irregular.

Thus for example shapes or patterns mimicking rivers and tributaries are included, even though they may meander in an apparently random way (as applies also to coastlines).

Whatever the nature of any shape(s) and/or patterns provided they should be easily detectable by touch. Thus they form a tactile region.

Bedding or bedwear should not substantially interfere with a person seeking to move a finger along such a given shape and/or pattern. For example the bedding or bedwear should not be unduly uncomfortable or heavy.

The present invention is therefore very different, for example, from US2015335853A, which discloses a weighted garment used to apply “deep pressure” to assist individuals with identified challenges of the nervous system.

The present invention does not require deep pressure to be applied. This is generally undesirable and can lead to poor circulation. On the contrary when using the present invention to try to get to sleep an individual should be able to lightly touch and trace a shape or pattern without deep pressure being applied by or to the individual.

It is also important to note that the present invention is concerned with bedwear and bedding, as well as methods of using them to help an individual get to sleep.

Thus it is not particularly concerned with everyday wear.

Furthermore, it does not require the tactile region to be located in a hidden/obscure manner on bedwear or bedding. On the contrary the tactile region is preferred to be prominent and easily accessible. This is very different from US2015273178A. which discloses a clothing system comprising integrated referred to as “fidgets”. Said system is said to be designed discretely for everyday wear so as to comfort wearers without them worrying about appearing strange to others.

The present invention is designed to allow a person to fall asleep more easily when lying down in bed. It is therefore different from inventions that are directed for seating. It is thus very different from the invention disclosed in US2017014595, whereby a customized anxiety therapy device is designed to encourage tranquil, seated, repetitive behaviour by Alzheimer's patients, dementia patients, and Autism Spectrum Disorder patients.

### BRIEF DESCRIPTION OF THE DRAWINGS

FIG. 1 is a top view of a tactile region made up of a series of concentrically arranged patterns;

FIG. 2 is a top view of a tactile region having a two-arm spiral arrangement;

FIG. 3 is a top view of a tactile region having a single-arm spiral arrangement of dots;

FIG. 4 is a top view of a tactile region formed of a spiral arrangement;

FIG. 5 is a top view of a tactile region of a further spiral arrangement;

FIG. 6 is a top view of a tactile region formed of another spiral arrangement;

FIG. 7 is a top view of a tactile region formed of a nested series of patterns and shapes arranged in successive ring-like structures around a central point;

FIG. 8 is a top view of a tactile region showing a stylized tree pattern;

FIG. 9 is a top view of a tactile region of another stylized tree;

FIG. 10 is a top view of a tactile region having two paths turning toward a central point;

FIG. 11 is a top view of a tactile region showing a series of stars joined by a line;

FIG. 12 is a top view of a tactile region showing a series of shapes that progressively diminish in size from right to left;

FIG. 13 is a top view of a tactile region in the shape of a sheep;

FIG. 14 is a top view of a tactile region of another representation of a sheep;

FIG. 15 is a top view of a tactile region of a series of sheep;

FIG. 16 is a top view of a tactile region of a series of stylized animals;

FIG. 17 is a top view of a tactile region of a series of stylized animals;

FIG. 18 is a top view of a tactile region of a series of animals arranged in concentric arrange;

FIG. 19 is a top view of a tactile region of large cloud images with smaller circular objects;

FIG. 20 is a top view of a tactile region of a series of clouds;

FIG. 21 is a top view of a tactile region having nested shapes;

FIG. 22 is a top view of a tactile region of nested lines;

FIG. 23 is a top view of a tactile region of a series of concentric circles;

FIG. 24 is a top view of a tactile region of a series of nested squares;

FIG. 25 is a top view of a tactile region of another series of nested squares; and

FIG. 26 is a top view of a tactile region of a further series of nested squares.

### DETAILED DESCRIPTION

The invention will now be described by way of example only, with reference to the associated figures.

In these examples various shapes and patterns are described. They are provided on bed wear or bedding where the shapes/patterns are either raised/recessed by at least 0.1 mm relative to surrounding material so as to form a tactile region. This therefore aids a user (defined as a wearer of bedwear or a person using bedding) in detecting the configuration by touch.

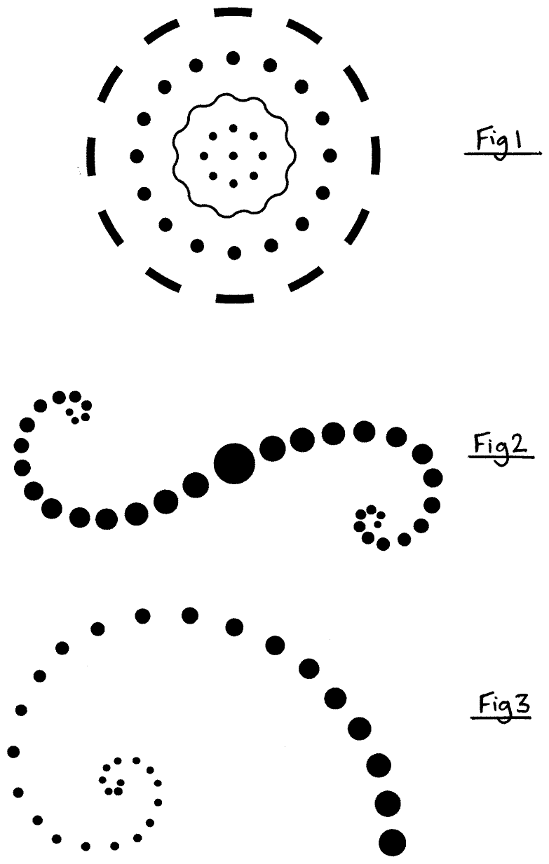

FIG. 1 shows a tactile region made up of a series of concentrically arranged patterns.

The outermost part of the arrangement is provided by a pattern of discontinuous curved elements that follow the circumference of a circle. The circle is centred on a point represented by the central dot (or disk) shown in the figure.

A user can trace a finger along the circumferentially arranged outermost elements and thus trace a generally circular path along the repeated pattern for as long or as short a time as desired. The path can be traced in a clockwise or anticlockwise manner (or even not at all) if the user chooses to move the finger to another level

In some cases this repeated generally circular motion may be enough to cause the user to fall asleep. It may distract the user from thoughts that are preventing/reducing the likelihood of sleep for sufficient time for the user to fall asleep.

The user is free to move the finger inwardly to the next concentric pattern, if desired. This is provided by a series of dots (discs). This provides a slightly different feel that can be detected by the user. Some users may prefer this feel.

The user is again free to move the finger around in the pattern and in contact with it for as few or as many times as desired (or even not at all and to skip to another level).

Again this can be done in a forward or reverse direction, which can here be considered as a clockwise or counter-clockwise direction.

Again there is a possibility that repeatedly moving the finger over the elements to trace and/or reverse a given path may induce sleep. The path can vary with the nature of the shape or pattern and it should be recalled that the examples are merely illustrative

In this example the user is also free to move the finger inwardly again to the next concentric pattern that is centred on a common central point. This is an undulating curve that forms a pattern that is generally cog shaped pattern. The finger can move along the wavy lines of the undulating curve for as long or as short a period as desired. This can be done in a generally clockwise and/or generally anticlockwise direction. Again there is a possibility that this will induce sleep. The undulating curve has a different feel from the patterns discussed above and can be distinguished by touch from said patterns. A user may for example prefer the undulating curve from other shapes and may move a finger around it until the user becomes fatigued and falls asleep

The next pattern shown in this figure is a further series of circumferentially aligned dots. This feels different from the wavy line and again a user can readily detect the difference by touch as the finger moves from the wavy line to the inner series of circumferentially arranged dots. level to another. The finger can move along the pattern of dots for as long or as short a period as desired and again this may help induce sleep.

At the centre of the arrangement of patterns is a single dot. This is useful aid to help a user identify the centre of the pattern by touch. In practice it is likely that a person seeking to get to sleep will spend the majority of the time moving a finger over one or more of the other patterns shown in this arrangement rather than touching the central dot. Indeed the central dot is a useful guide point, but is optional.

Thus the user is of course free to touch and/or move along one or more of the patterns or shapes shown in whatever manner the user wishes to and finds useful to induce sleep.

Preferably a user will start from the outermost pattern and move a finger inwardly to the innermost level However this is not essential and there are many alternatives. A user may skip one or more levels, may move the finger from an inner pattern to an outer pattern, etc

As discussed earlier a person seeking to sleep can repeat various movements based on touching the shapes or patterns and/or reverse them. It is preferred that there are a plurality of repeats and/or reversals of a given motion traced by a finger (e.g. a circular motion). Such action can aid the user in becoming fatigued getting to sleep. It may also induce a trance-like state that can help sleep.

Thus a user may move a finger over various shapes and/or patterns (or outlines thereof) in a given order and then repeat and/or reverse the process.

The variety of possibilities can itself be useful in distracting a person from thoughts that may prevent or delay sleep. The user may try a plurality of different possibilities and may with deeper and deeper focus on the very subtle differences between elements gradually fall asleep

In the arrangement shown in FIG. 1 the patterns shown are in a nested series. Here there are five patterns, if the central dot is included, or four if it is not.

It is of course possible to have fewer or more levels of nesting and also to have different patterns/motifs/shapes than is shown in this non limiting example.

Furthermore nesting need not use generally circular shapes/patterns that are circumferentially arranged round on a centre point. Any desired shapes or patterns can be nested

A series of nested generally polygonal shapes or patterns may for example be used. The arrangement may be generally triangular, generally square rectangular generally pentagonal, generally hexagonal, mixtures thereof, etc. The shapes or patterns may be continuous or discontinuous. Thus for example continuous or discontinuous lines (or dots) may be used to follow the outline of a polygon.

Indeed any nested arrangement can be used whether the shapes or patterns are the same, similar or different. Furthermore, if nesting is used, it is preferred that there be a symmetrical arrangement about a central point (as in FIG. 1), this is not essential.

FIG. 2 is a two-armed spiral arrangement made up of dots of various sizes. The dots get gradually smaller as a user traces a path of dots from a central point. The pattern shown can be considered to be a simulated fractal arrangement, because in theory if the pattern is repeated indefinitely the dots could get smaller and smaller and the pattern could continue infinitely. However in practice at a given level a user would not be able to distinguish between individual dots and thus at a given point the pattern of dots is stopped. Here there are 17 dots in each spiral that extend beyond the large central dot and get gradually smaller in size whilst still providing a spiral path for a finger to follow.

FIG. 3 is similar in many respects to FIG. 2, but here there is a single armed spiral arrangement of dots that is used to provide a path for a finger to follow. Furthermore here there are 32 dots. They get progressively smaller as the spiral moves inwards. It is of course possible to use other arrangements of shapes rather than dots to provide the spiral path for the finger to follow. It is also possible for the number of elements to differ.

FIG. 4 is another spiral arrangement but here the spiral is continuous and has a constant width. The coils of the spiral are spaced apart sufficiently so that the spiral shape and coils within it can be recognised by touch. It is preferred that there is a spacing of at least 2.5 mm, 5 mm, at least 10 mm or at least 15 mm between the coils of the spiral. The greater the spacing, the less likely it is that a finger following the spiral path will contact two adjacent coils at the same time.

The width of the spiral path is preferably less than 20 mm, less than 15 mm, less than 10 mm less than 5 mm or less than 2 mm. It may even for example be the width of a single thread of material. It is desired in any event that the that the path not be very wide because it would then be more difficult to detect and follow.

Thus ideally the width should be less than the width of a finger used to trace along the path.

Furthermore if the path is relatively narrow, more turns of the spiral can be fitted in a given area.

The length of the spiral is preferably at least 5 cm at least 10 cm at least 15 cm or at least 20 cm or at least 30 cm.

A long spiral allows a user to move a finger along it for a much longer time in a given direction than a much shorter one and this can help promote sleep. Indeed this applies whether or not the path is a continuous one as shown in this figure or is a discontinuous one (as shown in various other figures).

For example, FIG. 5 is a further spiral arrangement whereby a finger can follow a long path, but here the spiral is made up of discrete elements that make up the spiral path. Here the elements are rectangular blocks. A finger can trace along the blocks. In this embodiment the blocks form a path that gets gradually narrower as the finger moves from the outermost to innermost part of the spiral. This can add interest to a user who is following the spiral because the blocks are detectably different as the finger moves along the spiral.

However it is not essential that he blocks get narrower. It is preferred that even the widest block is less than 20 mm, less than 15 mm, less than 10 mm or less than 5 mm wide. The narrowest block may be 1 mm in width or less as long as it can be detected by touch.

As with other figures showing spiral arrangements it is preferred that the spacing between adjacent coils/turns of a spiral is at least 5 mm, at least 10 mm or at least 15 mm

In FIG. 5 there is a large dot at the centre of the spiral. (as for FIG. 1). This can help a user easily detect the of the spiral shown but is not essential

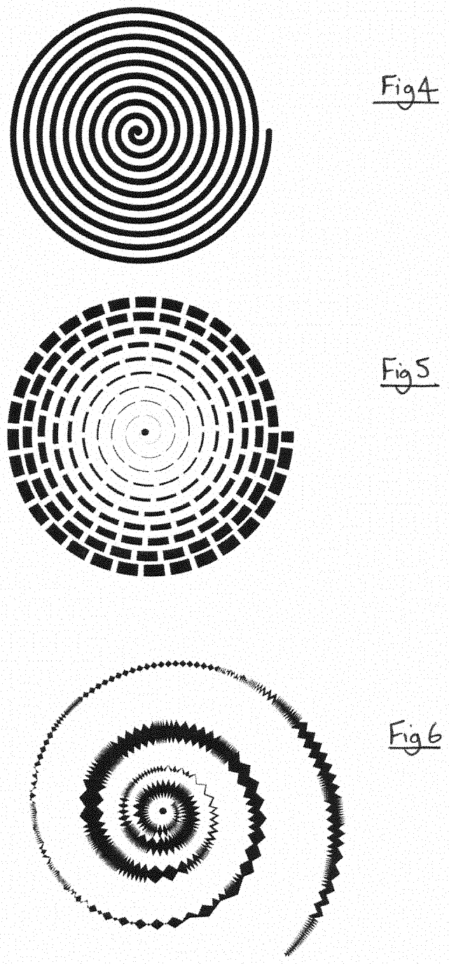

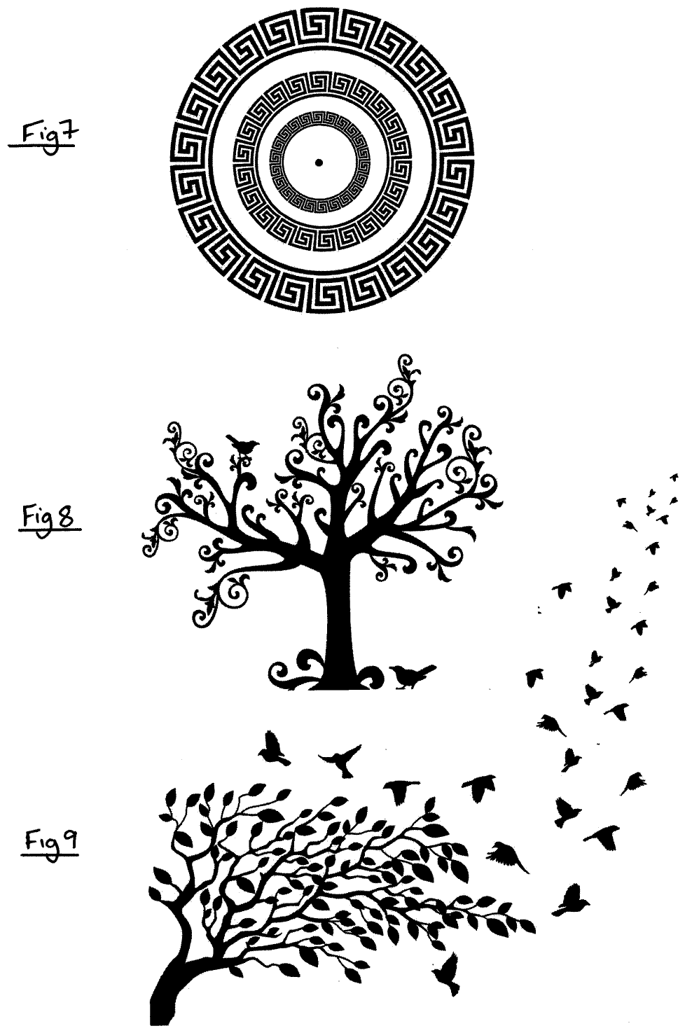

FIG. 6 is a yet further spiral arrangement but here the spiral has jagged edges that vary in nature and size along the length of the spiral. A user can trace the spiral path, but will note fairly random variations in the roughness/jaggedness of the spiral as the finger moves along it. Such random variations can be useful in that they can introduce an element of uncertainty as the finger moves along the spiral. This may help in distracting a user from thoughts that may otherwise prevent the user from getting to sleep. In theory such seemingly random variations could continue forever, as a spiral turns and turns (and as would be the case for a random fractal). In FIG. 6 the spiral may be considered as a simulated random fractal, given that manufacturing techniques and the sense of touch limit the number of variations along the spiral that would be provided in practice.

FIG. 7 is similar in some respects to FIG. 1 in that the is a nested series of patterns/shapes arranged in successive ring like structures around a central point

The outermost part of FIG. 7 is in the form of a of a meander pattern. Here a finger can trace a continual meandering path as it changes direction. The meander pattern shown is a regular meander pattern. Such patterns have been used to decorate pottery since the time of ancient Greek and Roman empires. They are still used today.

Meander patterns are very useful in the present invention in that they can provide a long pathway for a finger to trace in a small area. In FIG. 7 the meander pattern is in the form of a Greek key pattern. Other meanders are possible and may be regular or irregular meanders.

Close to the inside edge of each meander pattern shown in FIG. 7 there is a circle (or ring). This is optional and is not itself a meander. It can be considered to provide an inner border located close to the meander pattern.

As a user moves a finger inwards two or more meander patterns with borders can be found at increasingly small scales. Thus here there are three patterns that reduce in scale from the outermost to innermost pattern.

In theory the patterns could be repeated infinitely on a smaller and smaller scale, as is the case for fractals. However as discussed earlier this is not possible in practice, and the practical limit would be what the human fingertip can detect and differentiate. An arrangement shown of increasing small meander patterns nested within each other can be considered a simulated fractal arrangement of patterns. In the embodiment shown there is a central dot that is useful for orientation. However, as discussed earlier, this is optional.

FIG. 8 shows a stylised tree pattern. A bird is shown in the tree and another is shown next to the tree. The birds provide added elements of interest that can help in distracting a user from thoughts that may prevent sleep. The tree provides a trunk, branches from the trunk, branches of branches etc. This a finger can move along a wide variety of routes provide by the various branch points of the tree. The variety of routes that can be traced can help aid a user in getting to sleep. They can also help keep the user sufficiently interested so that the user does not give up on the task prematurely.

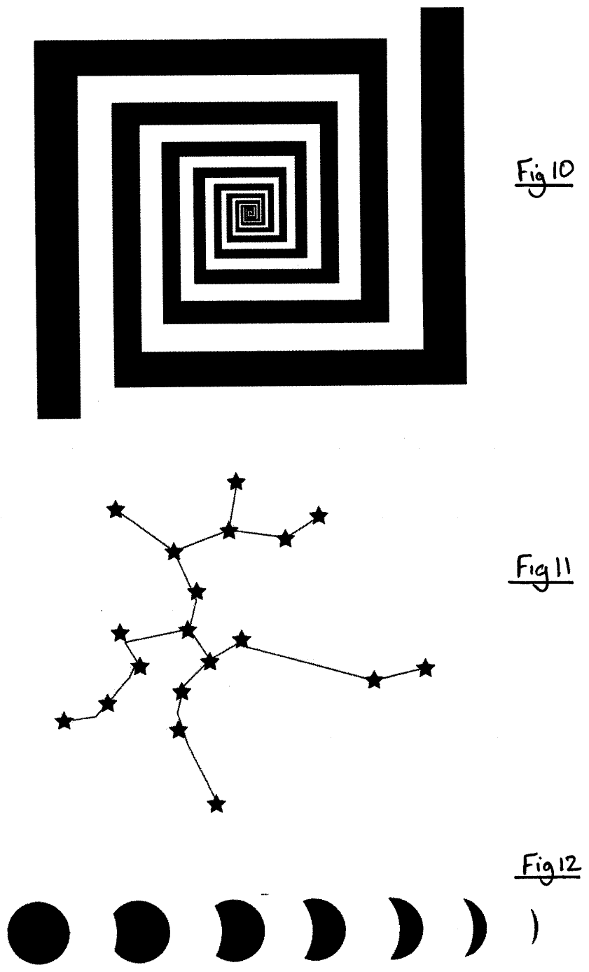

FIG. 9 is similar to FIG. 8 in that it is another stylised tree with birds associated with it. Here the tree is shown bent over to one side with a flock of birds shown ascending from the tree at that side. The birds get smaller and smaller as they get further away from the tree. Here a user can trace along branching of the tree and/or can trace the pattern of birds as the birds get smaller and smaller in a given direction. As with the other embodiments of the present invention the user is free to repeat and/or or reverse such actions as desired. This can promote sleep by leading to a state of fatigue whilst distracting the mind from thoughts preventing sleep

FIG. 10 shows an arrangement whereby two paths are shown with each path turning in on itself towards a central point. Each path can be considered as path that is linear but then turns through 90 degrees at regular intervals and gets narrower with each turn. This both paths converge towards a central point. The paths can be considered as two square spirals (also known as Ulam spirals).

This can be considered as a fractal arrangement given that in theory the spirals could continue forever at infinitely small scales. In practice, however, the arrangement can be consider as a simulated fractal arrangement, given that that, as discussed earlier, there are limitation on manufacturing techniques, as well as on the sense of touch that would allow a user to detect shapes or patterns at very small scales.

FIG. 11 shows a series of stars shown joined by lines. Many alternative arrangements could be shown so as to simulate clusters of stars or constellations. A user can use a finger to trace along the lines and stars. As with the other embodiments discussed herein, this can the user in getting to sleep. The arrangement shown include various branch points at which different routes can be taken by a finger.

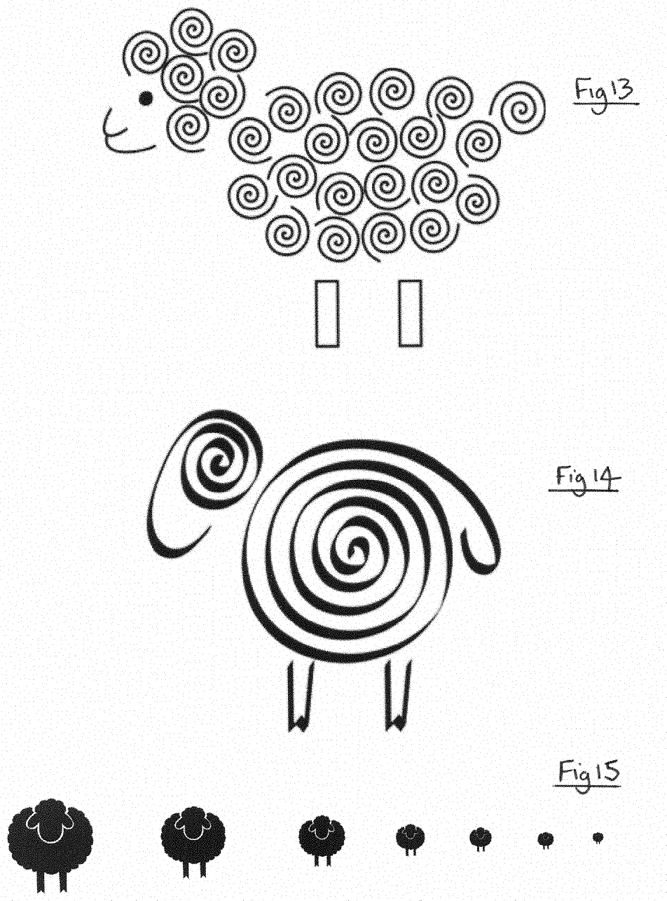

FIG. 12 show a series of shapes that get progressively smaller from right to left. The series begins with a filled circle and ends in a thin crescent. The series can be considered to schematically represent what could be seen remaining of one sphere/disc as another sphere/disc passes in front of it. Thus it mimics to some degree certain eclipses, as well as certain phases of the moon. A person moving a finger from right to left can trace the finger over the various shapes and detect the gradual change from a large circle to increasingly small shapes. As with other embodiment of the invention the sense of touch can help the person to get to sleep by helping distract the person's thoughts from those preventing sleep and helping the person become fatigued by performing a task involving moving a finger over all or part of a tactile region. This can be done one or more times. If, as here, a pattern is provided that provides various routes for a finger to trace is can help maintain interest in the task until a stage of deeper focus is reached.

The shapes can be spaced apart, but should be close enough so that a person having located one of the shapes by touch can easily find and move the finger over the other shapes. (In embodiments where a series of spaced apart shapes are provided, desirably each shape is less than 15 cm, less than 10 com, less than 5 cm or less than 5 cm from an adjacent shape)

FIGS. 13 to 15 are all schematic representations of a sheep (or ram), as an example of a design comprised of smaller tactile elements. FIGS. 13 and 14 include the common feature of having spiral that a user can trace a finger along (see the earlier discussion regarding spirals). They also include other shapes that help make up a schematic shape and can be detected by touch to provide a mental picture of the animal.

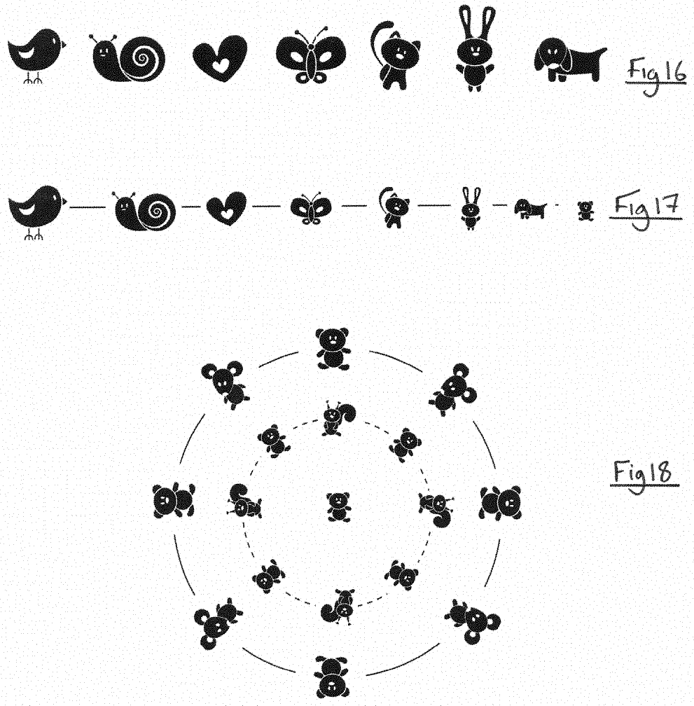

FIG. 15 show a sheep (or ram) getting smaller and smaller from left to right. A user can trace a pattern of an object getting smaller and smaller with a finger (or vice versa)

FIG. 16 shows a series of highly stylised animals and objects that vary from right to left. Indeed the images are emoji like in nature and particularly likely to appeal to children, teenagers, or young adults. A user can move a finger from one shape to the next and try to work out by touch what the shape represents. This can help distract the person from thoughts preventing sleep.

FIG. 17 is similar to FIG. 16, but the objects in FIG. 17 get progressively smaller from right to left and are separated by lines.

FIG. 18 show various schematic animal images being arranged in concentric rings. At the centre is a single animal image. This is to some degree analogous with FIG. 1 but replaces other shaped patterns with animals. This can be particularly useful for a person (especially a child or teenager) who likes animals trying to get to sleep

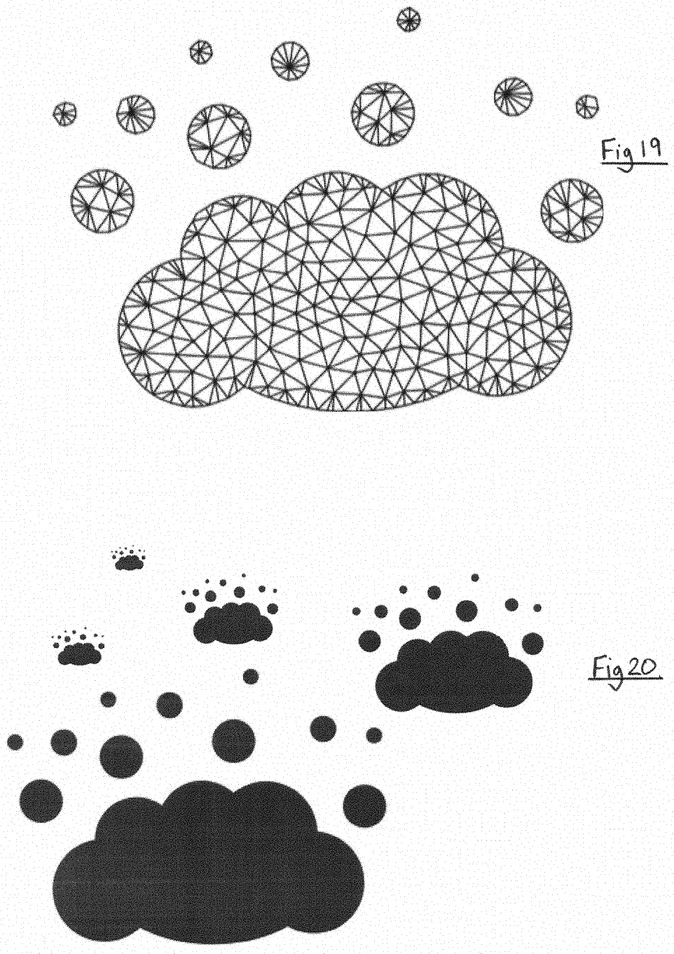

FIGS. 19 and 20 show large cloud images with smaller circular objects (that could represent mini-clouds or raindrops) located close to the clouds. The circular objects gradually reduce in size with distance away from the large clouds.

In FIG. 19 the clouds and circles contain various stitched lines that divide the cloud and circles into numerous generally triangular parts. A finger can be used not only to identify the clouds and circles but also to move over the internal lines, find nodes where lines intersect, identify triangular shapes, etc. Here there is a patchwork-like arrangement of internal lines that provide many possible routes for a finger to move along. Typically the lines would be formed of threads

In FIG. 20 the clouds and circles are blocks of material. Furthermore there are numerous repetitions of a clouds and associated circles at different relative sizes. Thus a user can move a finger over a tactile region where the images are present and try to identify the different sizes and shapes. Again, this can help aid sleep by distracting the person from thoughts that might present sleep and in providing a task involving the sense of touch that is of sufficient interest for a user to engage in it until the user becomes more and more deeply focused and may fall asleep.

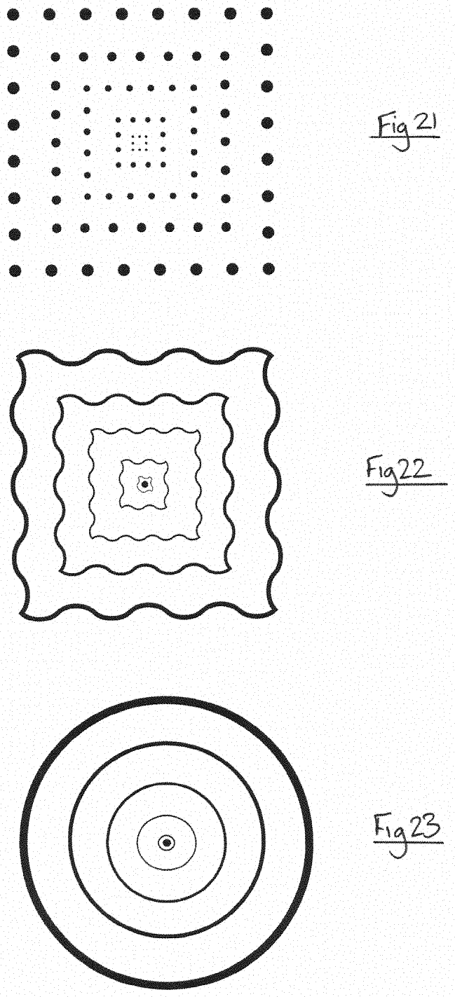

FIG. 21 to FIG. 26 are similar to some of the earlier figures in that they provide nested shapes/patterns whereby a user can move from an outer level to one or more inner levels. At each level the user can move a finger around a path provided by a given shape or pattern. This can help the user to get to sleep.

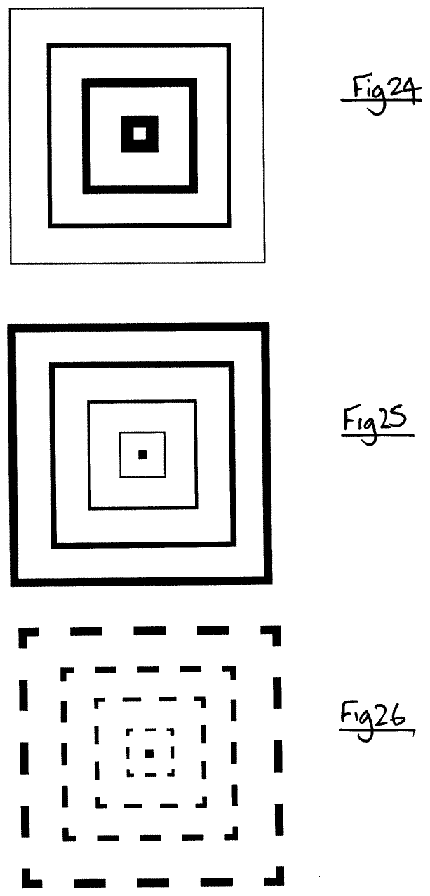

In FIG. 21 square nested patterns based on a series of dots are provided. FIG. 26 is generally similar to FIG. 21, but uses short lines rather than dots. FIGS. 22 to 25 all have nested shapes formed by continuous lines. The shapes vary in nature from nested square shapes (see FIGS. 24 and 25), nested generally square shapes (see FIG. 22, where wavy lines rather than straight lines are used) and nested circles/rings (see FIG. 23)

A person seeking to fall asleep may optionally listen to a sound recording discussing using the sense of touch to feel and move over/along one or more shapes, configurations or patterns that can help promote sleep.

Thus, for example, if the tactile region comprises a spiral pathway shaped for a finger to move along, the recording may discuss gradually moving the finger along the spiral. It may also discuss repeating or reversing this action.

The voice recording may be timed to synchronise with a timing of the movement likely to promote sleep. For example, it may discuss moving a finger along an outer coil of a spiral may then discuss progressively moving it along inner coils in a manner that corresponds with a desired timing for the movement.

The voice recording may if desired be accompanied by relaxing music or sounds (e.g. the sound of waves, of birds singing, etc.).

The voice recording may be provided on a data carrier (e.g. in the form of a recording stored by a digital or analogue memory).

The invention also includes a device set up to transmit such a recording. The device may for example be a mobile phone/or computer that includes the voice recording, or that comprises an “app” that links to the recording. The device may be any device with a sound input system that is arranged to transmit the recording.

The invention also includes kits. For example, it includes a kit comprising bedding or bedwear according to the invention and instructions (e.g. voice instructions) for using the bedding bedwear or patch in a method of the invention.

As explained above, voice instructions may be provided via a data carrier and/or via a device set up to transmit such a recording. Thus the carrier and/or device may be included in the kit.

Another example of a kit of the present invention is a kit that allows existing bedding or bedwear to be converted to provide bedding or bedwear of the present invention.

This may include a tactile region (e.g. in the form of a patch) that can be secured to existing bedding or bedwear to transform it to bedding or bedwear of the present invention. If desired, the kit may also include instructions for securing the tactile region to existing bedding or bedwear and/or means for securing the tactile region to existing bedding or bedwear (e.g. adhesive, sewing thread, etc.)

1. A method of aiding sleep; whereby a person seeking to sleep uses a finger to touch and move the finger over at least part of a tactile region located on bedwear that the person is wearing, or on bedding present on a bed, in a manner that can promote sleep.

2. A method according to claim 1; wherein the tactile region comprises one or more of the following: a spiral shape or pattern, a group of nested or concentric shapes or patterns, a series of shapes or patterns that get progressively smaller or larger in a given direction, a series of shapes or patterns that incorporate variation between the elements, a line arranged to provide a repeated motif or pattern, a shape that includes a plurality of branches, a generally fractal shape, a series of shapes or patterns having a common theme.

3. A method according to claim 1 wherein the tactile region, shape(s) or patterns(s) is/are raised or recessed by at least 0.1 mm (e.g. at least 1 mm 2 mm at least 5 mm or at least 10 mm).

4. A method according to; claim 1 wherein the tactile region comprises one or more shapes or patterns that get progressively smaller and/or more detailed.

5. A method according to claim 4; wherein the tactile region comprises a shape or pattern that is self-similar at a level of at least three repeats or iterations.

6. A method according to claim 1 wherein the tactile region comprises a simulated fractal shape or pattern.

7. A method according to; claim 1 wherein the person repeatedly moves a finger in a given manner over all or at least part of the tactile region until the person develops focus and concentration on the element being explored, and/or becomes fatigued and/or distracted from thoughts that help keep the person awake, and is thereby more likely to sleep.

8. A method according to; claim 1 wherein the tactile region is located on bedwear worn by the person seeking to sleep.

9. A method according to claim 8; wherein the tactile region is provided at a front, side, arm, leg and and/or torso area of bedwear at a location so as to be easily accessible to a finger of the person wearing the bedwear in bed and seeking to sleep.

10. A method according to claim 1; wherein the method includes the step of a person listens to a sound recording instructing the person to touch the tactile region and move along it in a given manner (e.g. wherein the recording further includes music).

11. A method according to claim 1; wherein the tactile region comprises a path or pathway for the finger to trace.

12. A method according to claim 11; wherein the path or pathway is at least 1 cm, 2 cm, 5 cm, at least 10 cm, at least 20 cm or at least 30 cm long.

13. A method according to claim 11; wherein the tactile region provides a path or pathway for the finger to trace that is less than 2 cm wide.

14. A method according to claim 11; wherein the tactile region provides a continuous path or pathway for the finger to trace.

15. A method according to claim 1; wherein the tactile region is located only at a discrete region of bedwear or bedding.

16. A method according claim 1; wherein the tactile region represents less than 25%, 10%, less than 5% or less than 1% of the area of a given surface of the bedding or bedwear.

17. A method according to claim 1 when used by a person to get to sleep in less than 60 minutes, less than 30 minutes, less than 15 minutes, less than 10 minutes or less than 5 minutes from the person commencing the method.

18. Bedwear or bedding that comprises a tactile region as described in claim 1.

19. A method comprising modifying existing bedding or bedwear that does not have the tactile region described in claim 11; whereby the method includes adding said region in the form of a patch to said bedding or bedwear.

20. (canceled)

21. (canceled)

22. A kit comprising bedding or bedwear according to claim 18, wherein the kit comprises instructions for using the bedding bedwear or patch.

23. (canceled)

24. (canceled)

25. (canceled)
